(19) United States
(12) Patent Application Publication
(10) Pub. No.: US 2005/0106607 A1
(43) Pub. Date: May 19, 2005

(54) Biochip containing reaction wells and method for producing same and use thereof

(75) Inventors: Li-Te Yin, Taipei City (TW); Chih-Yu Hu, Kaohsiung City (TW); Chao-Yun Tsao, Taipei City (TW); Su-Fung Chiou, Keelung City (TW); Jia-Huey Tsao, Taoyuan City (TW)

Correspondence Address: BACON & THOMAS, PLLC, 625 SLATERS LANE, FOURTH FLOOR, ALEXANDRIA, VA, 22314, US

(73) Assignees: Industrial Technology Research Institute, Hsin Chu (TW); Co-Wealth Medical Science & Biotechnology INC., Taipei County (TW)

(21) Appl. No.: 10/965,792

(22) Filed: Oct. 18, 2004

(30) Foreign Application Priority Data
Nov. 19, 2003 (TW) 92132395

(51) Int. Cl. C12Q 1/68

(52) U.S. Cl. 435/6

(57) ABSTRACT

This invention is provided a biochip containing splitable reaction wells and method for producing same and application thereof. The invention avoids cross contamination or interference between different samples on the same biochip. In addition, the soft polymer film removed from reaction wells may be reused on different substrates, thereby saving the cost of experiment or clinical testing.

### BACKGROUND OF THE INVENTION

1. Field of the Invention

The present invention relates to biochips where the chip contain a plurality of reaction wells. The biochip provided herein may be used in microarray analysis where the situation of cross contamination or interference between samples on the same biochip is effectively avoided.

2. Description of Related Art

As the work of human genome mapping came to a close in April 2003, biotechnology stepped into the age of proteomics from genomics. In light of the meteoric growth of gene and protein related data, biochip that allows mass and rapid screening has become an indispensable research and detection tool. Biochips are not clearly defined or categorized. It typically refers to precise, miniaturized device using silicon chip, glass or polymer as substrate and integrating micro technologies in the fields of mechanico-electrical, opto-electrical, chemistry, biochemistry, medical engineering and molecular biology. Biochips may be used in medical testing, environmental testing, food testing, new drug development, basic research, national defense, and chemical synthesis. Biochips are classed into gene chip, protein chip, and lab-on-a-chip on the market, of which gene chip is more mature in terms of development. Biochips referred to by the research community or biotech industry in Taiwan are primarily gene chips. Gene chips are fabricated by rapidly spotting pre-synthesized DNA probes into high-density array (2500 spots/cm²) on substrate surface using microarray technique and robotic arm, and the tested samples are typically cDNA target. For testing, the substrate and the sample undergo hybridization, in which the target nucleic acid in the sample would hybridize to the spot of probe on cDNA microarray substrate containing complementary nucleic acid sequence. After washing off unhybridized nucleic acid in the sample, the spots having hybridization reaction are marked down. As such, cDNA microarray can analyze the gene expression pattern of sample in one test. Protein chips use protein as probes, which are immobilized on substrate surface using microarray technique. Through antigen-antibody reaction, protein chips may be applied in the analysis of protein expression pattern or screening of new drug candidate. Lab-on-a-chip is designed according to needs where different reactions take place on a microchip. Currently biochemical reactions that may be carried on lab-on-a-chip include polymerase chain reaction (PCR), nucleic acid sequencing reaction, microfluidics, electrophoresis, mass spectrography, antigen-antibody binding, and regular enzymatic reaction.

Microarray chips commonly use DNA or protein for medical testing or experimental analysis. Regular microarray chip can only be used in multiple tests of a single sample. If multiple samples are analyzed on a single chip, there will be cross contamination between samples, which limits the capability of the chip to perform simultaneously testing of multiple samples in multiple regions. Thus the development of a kind of biochip having reaction wells and on which the detection of multiple samples may be performed is a topic that warrants attention.

### SUMMARY OF THE INVENTION

In addressing the drawback of biochip that cannot perform analysis of a plurality of samples simultaneously, the present invention discloses biochips containing reaction wells and method for producing same and use thereof.

The object of the present invention is to provide a biochip containing reaction wells, comprising a substrate having a surface; a polymer film having a plurality of holes adhered to the surface of the substrate to form wells; wherein the material of the polymer film is selected from a group consisting of polydimethylsiloxane(PDMS), polystyrene(PS), polypropylene(PPR) and mixture thereof; and a plurality of probes immobilized in the wells on the substrate.

Another object of the present invention is to provide a method for fabricating biochip comprising the steps of: providing a polymer film; wherein the material of the material of the polymer film is selected from a group consisting of polydimethylsiloxane(PDMS), polystyrene(PS), polypropylene(PPR) and mixture thereof; cutting the polymer film to produce a plurality of holes; providing a substrate; subjecting the substrate to surface treatment; adhering the polymer film on the substrate to form a plurality of wells on the substrate; and immobilizing probes in the wells on the substrate.

Yet another object of the present invention is to detect the presence of analytes in a sample, the method comprising the steps of: contacting (a) a biochip having a plurality of probes that specifically reacts with the analytes, with (b) a sample suspected of comprising the analytes under the conditions sufficient for reacting the analytes and the probes on the biochip to produce reaction complexes; removing the polymer film from the substrate; and detecting the presence of the reacting complexes on the surface of the biochip; whereby the presence of the analytes in the sample are detected.

In one embodiments of the present invention, the method may further comprise the step of sample labeling, wherein samples are labeled with fluorophore or chromophore (e.g. green fluorescent dye (Cy3) or red fluorescent dye (Cy5)) to facilitate the detection of signals from the chip.

The biochip containing reaction wells provided herein may be used in microarray analysis where the situation of cross contamination or interference between samples on the same biochip is effectively avoided. Similarly through a plurality of groove-shaped holes on the reaction wells, different samples may be analyzed on the same chip, thereby greatly enhancing the efficiency of the microarray chip and diversity of detection. In addition, the polymer film may be reused on different substrates, thereby saving the cost of experiment or clinical testing. The removal of film also helps maintain standardization in subsequent detection of chip signals.

### BRIEF DESCRIPTION OF THE DRAWINGS

FIG. 1 shows the schematic diagram of biochip with reaction wells according to the present invention.

FIG. 2 shows the flow chart of fabricating biochip with reaction wells according to the present invention.

FIG. 3 shows the flow chart of a detecting method using biochip with reaction wells according to the present invention.

FIG. 4 shows the detecting results using biochip with reaction wells according to the present invention.

### DETAILED DESCRIPTION OF THE INVENTION

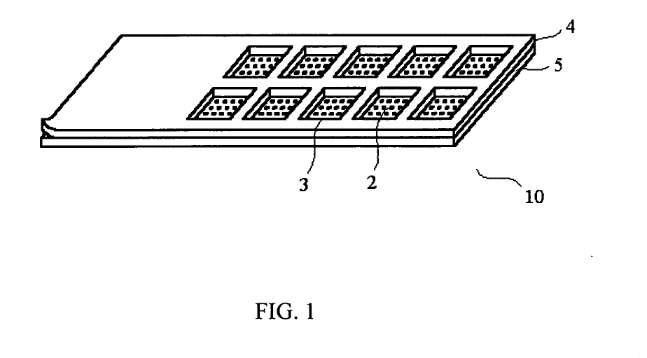

The present invention provides a biochip 10 as shown in FIG. 1, comprises a substrate 5 having a surface; a polymer film 4 having a plurality of holes adhered to the surface of the substrate 5 to form the wells 3; and a plurality of probes 2 are immobilized in the wells 3 on the substrate 5, wherein the polymer film 4 is splitable from substrate 5, and may be used repeatedly.

The material of the polymer film may be made of, for example, but not limited to, polydimethylsiloxane (PDMS), polystyrene (PS), polypropylene (PPR) and mixture thereof, preferably PDMS Preferably, the polymer film has a thickness about 1-2 mm. The number and shape of holes on the polymer film may vary according to actual needs without particular restrictions.

The substrate may be made of, for example, but not limited to, quartz, glass, plastic, silicon or polymer. And the probes immobilized in the wells on the substrate are biomaterials including, but not limited to, DNA, protein, or cells.

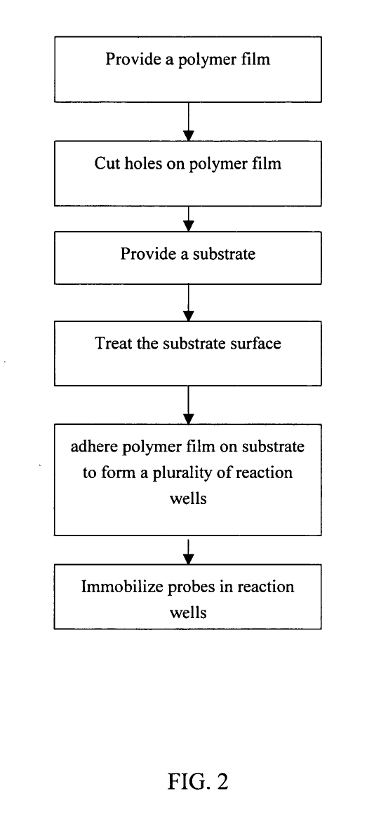

As shown in FIG. 2, the method for fabricating biochip containing reaction wells comprises the steps of: providing a polymer film; cutting the polymer film to produce a plurality holes; providing a substrate; subjecting the substrate to surface treatment; adhering the polymer film on the substrate to form a plurality of reaction wells on the substrate; and immobilizing probes in the wells on the substrate. The aforesaid steps are described in detail below: first produce flexible and freely twistable polymer film with dichloro dimethylsiloxane, dihydroxyl dimethylsiloxane, styrene or propylene monomer via condensation reaction; the polymer film is preferably polydimethylsiloxane (PDMS); next cut the polymer film to produce a plurality of holes using imprinted mold made by computer numeric controller (CNC), or by means of casting, soft lithography, or stereo lithography. The specification and quantity of reaction wells may vary depending on detection need in the cutting procedure. Subsequently, carry out the surface treatment of substrate, in which biochemically immobilized material, such as polystyrene-co-maleic-anhydride (PSMA), polystyrene (PS), nitrocellulose (NC), polyvinylidenedifluoride (PVDF), gold or nickel is coated on substrate surface. The substrate may be made of quartz, glass, plastic, silicon or polymer. Next adhere the polymer film on the surface-treated substrate to form a plurality of reaction wells on the substrate. The adhering of the polymer film with substrate is achieved by physically adhering the polymer film to the substrate without the aid of other chemicals. Finally, immobilize probes in the reaction wells to finish the fabrication of biochip containing reaction wells. The probes may be DNA, protein, or cells.

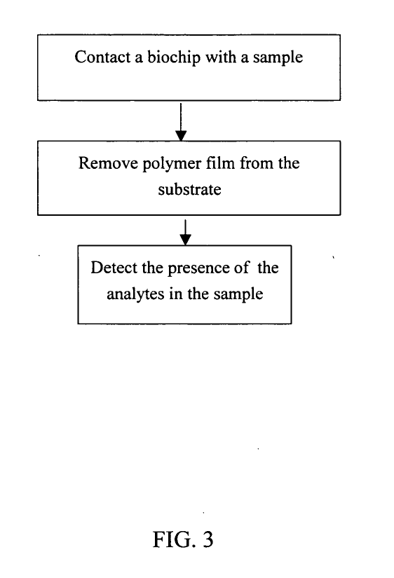

The detecting method applying the aforesaid biochip as shown in FIG. 3 comprises the steps of: contact a biochip with a sample suspected of comprising said analytes, wherein the biochip having reaction wells with probes immobilized therein. Subsequently, the reactions of the analytes and the probes will occur under a suitable conditions. Then, remove the polymer film from the substrate. Finally, detect the presence of the analytes in the sample. The biochip containing reaction wells is fabricated by immobilizing probes in reaction wells using microarray technique; the probes include DNA, protein, or cells.

In one embodiment of the present invention, the samples may be further labeled with fluorophore or chromophore (e.g. Cy3 or Cy5). The reaction between samples and probes may be hybridization, antigen-antibody reaction or other specific reactions between biomolecules. After the reaction between samples and probes, the polymer film on the substrate is removed from the substrate surface to facilitate the subsequent procedure of signal detection, in which scanning device is used to detect the fluorophore or chromophore labeled on samples that have reacted with the probes. The detection results depicting the imaging of fluorescence intensity or color development are subjected to computer analysis to define the expression pattern of the sample.

The advantages of the present invention are further depicted with the illustration of examples, but the descriptions made in the examples should not be construed as a limitation on the actual application of the present invention.

### EXAMPLE 1

### Preparation of Biochip Containing Reaction Wells

Use computer numeric controller (CNC) to produce stainless imprinted mold and use such mold to cut holes on polydimethylsiloxane (PDSM) polymer film (10 holes in total (5×2)); the size of the film obtained thereof is the same as commercially available slide (7.5 cm×2.5 cm) and size of holes thereon is 0.7 cm×0.65 cm with 0.4 cm of space between adjacent holes and between holes and the edge of film. In addition, take a piece of commercially available slide and coat its surface with polystyrene-co-maleic-anhydride (PSMA). Join the PDMS film with the surface-treated slide according to the orientation of the holes to obtain a reaction wells. Next immobilize probes (including DNA, protein, or cells for the working of the biochip) in reaction wells to obtain a biochip.

### EXAMPLE 2

### Applying Biochip Containing Splitable Reaction Wells in Testing

Deposit the test samples directly in the reaction wells on biochip shown in Example 1. After 2 hours of reaction, remove non-specific binding samples. Next inject fluorescent signal molecules into the reaction wells on biochip shown in Example 1 to let the signal molecules react with probe-bound samples for 1 hour. Wash the reaction wells afterwards to remove unbound signal molecules. Next remove polymer film on biochip surface, and then place the biochip in fluorescence scanner to detect the signals of samples that have reacted with microarrayed probes. Finally computer analyze the correlation between the fluorescence imaging and molecular probes to learn about the expression pattern of samples.

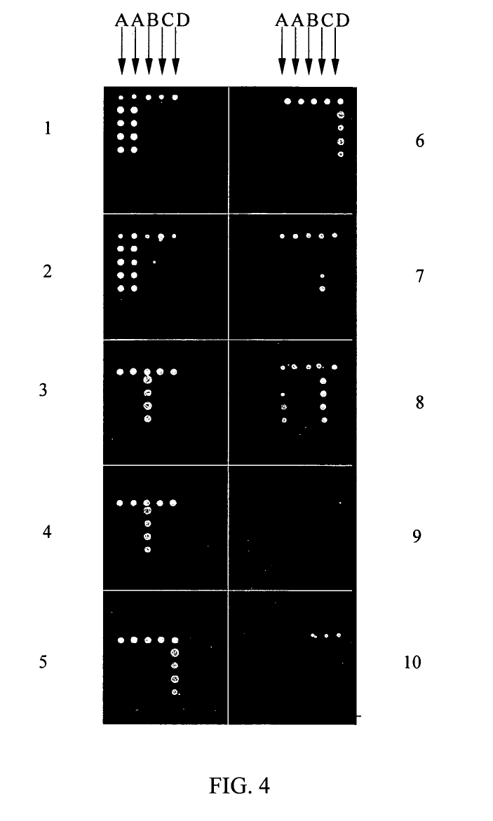

FIG. 4 shows the detecting results of biochip provided herein. The types of molecular probes immobilized on individual reaction wells are as follows: A is cat dander allergen (from Center Laboratories Inc.); B is peanut allergen (from Center Laboratories Inc.); C is dust mite allergen (from Center Laboratories Inc.); and D is yolk allergen (from Nelco Corporation). Dilute the aforesaid allergen solutions with 0.1M sodium carbonate solution to proper ratio and spot the diluted allergen solutions in respective reaction wells on the biochip using microarray technique where each spot is 20 nl in size. Then deposit different samples (serum of allergy patients) in the volume of 30 μl each onto respective reaction wells to let them react with probes, whereas each sample is subject to two-duplicate test, i.e. deposit the first test sample in the 1st and 2nd reaction wells; deposit the second test sample in the 3rd and 4th reaction wells; deposit the third test sample in the 5th and 6th reaction wells; deposit the fourth test sample in the 7th and the 8th reaction wells; and deposit 0.1M phosphate buffered saline in the 9th and 10th reaction wells as control. The results indicate that the probe and sample binding signals in different reaction wells did not interfere with each other.

The aforesaid test demonstrates that the biochip containing splitable reaction wells provided herein may be used in detecting a plurality of samples and there is no interference between sample signals.

To sum up, using the splitable reaction wells on a substrate (e.g. microarray chip) disclosed herein to detect the same or different test samples can effectively avoid the cross contamination or interference between samples, and through a plurality of groove-shaped holes on the reaction wells, different samples may be tested on the same chip, which significantly enhance the efficiency and detection diversity of microarray chip. Moreover, the polymer film removed from the chip may be recycled and used on different substrates, thereby lowering the product cost.

The preferred embodiments of the present invention as disclosed above are not meant to limit this invention. All modifications and alterations made by those familiar with the skill without departing from the spirits of the invention and appended claims shall remain within the protected scope and claims of the invention.

1. A biochip comprising:
a substrate having a surface;
a polymer film having a plurality of holes adhered to the surface of said substrate to form wells; wherein the material of said polymer film is selected from a group consisting of polydimethylsiloxane(PDMS), polystyrene(PS), polypropylene(PPR) and mixture thereof; and
a plurality of probes immobilized in said wells on the substrate.

2. The biochip according to claim 1, wherein the material of said polymer film is polydimethylsiloxane (PDMS).

3. The biochip according to claim 1, wherein the thickness of said polymer film is about 1˜2 mm.

4. The biochip according to claim 1, wherein said polymer film is splitable from said substrate.

5. The biochip according to claim 1, wherein said substrate is made of quartz, glass, plastic, silicon or polymer.

6. The biochip according to claim 1, wherein said probes are DNA, proteins, or cells.

7. A method for fabricating a biochip comprising the steps of:
providing a polymer film; wherein the material of said polymer film is selected from a group consisting of polydimethylsiloxane(PDMS), polystyrene(PS), polypropylene(PPR) and mixture thereof;
cutting said polymer film to produce a plurality of holes;
providing a substrate;
subjecting said substrate to surface treatment;
adhering said polymer film on said substrate to form a plurality of wells on the substrate; and
immobilizing probes in said wells on the substrate.

8. The method according to claim 7, wherein the material of said polymer film is polydimethylsiloxane (PDMS).

9. The method according to claim 7, wherein the thickness of said polymer film is about 1˜2 mm.

10. The method according to claim 7, wherein the way for cutting said polymer film to produce a plurality of holes comprises imprint, casting, soft lithography, or stereo lithography.

11. The method according to claim 7, wherein said substrate is made of quartz, glass, plastic, silicon or polymer.

12. The method according to claim 7, wherein said surface treatment of said substrate involves coating of substrate with immobilized material.

13. The method according to claim 12, wherein said immobilized material is polystyrene-co-maleic-anhydride (PSMA), polystyrene (PS), nitrocellulose (NC), polyvinylidenedifluoride (PVDF), gold, or nickel.

14. The method according to claim 7, wherein said molecular probes are DNA, proteins, or cells.

15. A method for detecting the presence of analytes in a sample, said method comprising:
contacting (a) a biochip according to claim 1 having a plurality of probes that specifically reacts with said analytes, with (b) a sample suspected of comprising said analytes under the conditions sufficient for reacting said analytes and said probes on said biochip to produce reaction complexes;
removing said polymer film from the substrate; and
detecting the presence of said reacting complexes on surface of the said biochip; whereby the presence of said analytes in said sample are detected.
